(19) United States
(12) Patent Application Publication
(10) Pub. No.: US 2022/0096832 A1
(43) Pub. Date: Mar. 31, 2022

(54) MEDICAL ELECTRICAL STIMULATION DEVICE WITH DYNAMIC IMPEDANCE

(71) Applicant: Medtronic, Inc., Minneapolis, MN (US)

(72) Inventor: Erik J. Peterson, Fridley, MN (US)

(21) Appl. No.: 17/036,928

(22) Filed: Sep. 29, 2020

(51) Int. Cl. A61N 1/36; A61N 1/05; A61N 1/08; A61N 1/375

(52) U.S. Cl. CPC A61N 1/3614; A61N 1/0529; A61N 1/36082; A61N 1/3754; A61N 1/086

(57) ABSTRACT

In some examples, an implantable medical device (IMD) includes a stimulation generator and/or sensing circuitry configured to generate electrical stimulation for delivery to or sensing the state of a patient via electrode coupled to the IMD; interconnect circuitry configured to transport the electrical stimulation from the stimulation generator to the lead, the interconnect circuitry comprising: a feedthrough capacitor; and one or more components and a switch that are collectively electrically connected in parallel with the feedthrough capacitor; and processing circuitry configured to selectively close the switch based on a magnetic resonance imaging (MRI) status of the IMD.

### TECHNICAL FIELD

The disclosure relates to medical devices for delivering electrical stimulation therapy.

### BACKGROUND

Medical devices may be used to treat a variety of medical conditions. Medical electrical stimulation devices, for example, may deliver electrical stimulation therapy to a patient via implanted electrodes. Electrical stimulation therapy may include stimulation of nerve, muscle, or brain tissue, or other tissue within a patient. An electrical stimulation device may be fully implanted within the patient. For example, an electrical stimulation device may include an implantable electrical stimulation generator and one or more electrodes. In some implementations, the electrodes may be carried by one or more implantable leads or include some one or more electrodes carried by leads and one or more electrodes no carried by leads.

### SUMMARY

The present disclosure relates to implantable medical devices with dynamic impedance for promoting magnetic resonance imaging (MRI) tolerance. During normal operation, i.e., when the an implantable medical device (IMD) is not in the presence of MRI energy, the IMD may deliver electrical stimulation to a patient and/or sense electrical signals of the patient via one or more electrodes, some or all of which may be carried, in some examples, on one or more conductive leads. The conductive leads may be connected to components of the IMD (e.g., a stimulation generator and/or sensing circuitry) via interconnect circuitry, such as one or more feedthrough components, which may include one or more feedthrough capacitors. An MRI system may be configured to perform an MRI scan on a patient having an IMD. During the MRI scan, a voltage may be induced in the one or more electrodes and/or the conductive leads by the MRI system as a result of electric fields produced by RF excitation during an MRI scan by an MRI scanner. Such RF excitation may cause heating of the electrode(s), conductive lead(s), circuit components, and/or a housing of the IMD. RF excitation may also cause unwanted voltages to be coupled onto electrodes, causing undesired stimulation.

The amount of heating or stimulation generated in the IMD as a result of the MRI scan may have a negative relationship with an impedance of the interconnect circuitry. For instance, lower shunting impedances to the IMD case may result in relatively lower heating while higher shunting impedances may result in relatively higher heating or stimulation. Therefore, interconnect circuitry with lower shunting impedances may reduce undesirable heating during MRI scans. However, lower shunting impedances of the interconnect circuitry may present one or more disadvantages. As one example, the impedance of the interconnect circuitry may have a negative relationship with an amount of energy used to deliver a particular level of stimulation. For instance, lower shunting impedances in the interconnect circuitry may result in an increased amount of energy (e.g., battery power) being expended to deliver a particular level of stimulation as compared with higher impedances. Therefore, interconnect circuitry with higher shunting impedances may enable more efficient operation. As another example, the impedance of the interconnect circuitry may have a positive relationship with a quality of sensing. For instance, lower shunting impedances in the interconnect circuitry may result in a decrease in common-mode signal rejection as compared with higher impedances. Therefore, interconnect circuitry with lower impedances may enable improved sensing functionality.

In accordance with one or more aspects of this disclosure, an implantable medical device (IMD) may be configured to dynamically adjust an impedance of interconnect circuitry based on a magnetic resonance imaging (MRI) status of the IMD. For instance, in normal operation, the interconnect circuitry may have a first impedance. Responsive to determining that the IMD has been placed in an MRI mode or that an MRI field is detected, the IMD may configure the interconnect circuitry such that the interconnect circuitry has a second shunting impedance that is lower than the first impedance. In this way, the IMD may benefit from a high shunting interconnect circuitry impedance (e.g., reduced energy consumption, improved sensing quality) while also selectively benefiting from a low shunting interconnect circuitry impedance (e.g., reduced MRI heating).

In one example, an IMD includes a stimulation generator configured to generate electrical stimulation for delivery to a patient via a lead coupled to the IMD; interconnect circuitry configured to deliver the electrical stimulation from the stimulation generator to the lead; and processing circuitry configured to adjust an impedance of the interconnect circuitry based on a MRI status of the IMD.

In another example, a method includes generating, by a stimulation generator of an IMD, electrical stimulation for delivery to a patient via a lead coupled to the IMD; transporting, by interconnect circuitry, the electrical stimulation from the stimulation generator to the lead, the interconnect circuitry comprising: a feedthrough capacitor; and one or more components and a switch that are collectively electrically connected in parallel with the feedthrough capacitor; and selectively operating the switch based on a MRI status of the IMD.

In another example, a computer-readable storage medium stores instructions that, when executed, cause processing circuitry of an IMD to: cause a stimulation generator to generate electrical stimulation for delivery to a patient via a lead coupled to the IMD, the electrical stimulation being transported from the stimulation generator to the lead via interconnect circuitry, the interconnect circuitry comprising: a feedthrough capacitor; and one or more components and a switch that are collectively electrically connected in parallel with the feedthrough capacitor; and selectively operate the switch based on a MRI status of the IMD.

The details of one or more aspects of the disclosure are set forth in the accompanying drawings and the description below. Other features, objects, and advantages will be apparent from the description and drawings, and from the claims.

### BRIEF DESCRIPTION OF THE DRAWINGS

FIG. 1 is a conceptual diagram illustrating an example medical device system.

FIG. 2 is a conceptual diagram illustrating the example implantable medical device of shown in FIG. 1.

FIG. 3 is a conceptual diagram illustrating the example medical device programmer shown in FIG. 1.

FIG. 4 is a schematic diagram illustrating one example of interconnect circuitry with adjustable impedance, in accordance with one or more aspects of this disclosure.

FIG. 5 is a flow diagram illustrating an example technique for dynamically adjusting an impedance of an implantable medical device, in accordance with one or more aspects of this disclosure.

FIG. 6 is a flow diagram illustrating an example technique for dynamically adjusting an impedance of an implantable medical device, in accordance with one or more aspects of this disclosure.

FIG. 7 is a conceptual diagram illustrating an example medical device system.

### DETAILED DESCRIPTION

As described above, some examples of the disclosure are directed to medical device systems and techniques involving features to promote stimulation and/or sensing performance and compatibility with magnetic resonance imaging (MRI) scans. MRI has been developed as an imaging technique adapted to obtain both images of anatomical features of human patients as well as some aspects of the functional activities of biological tissue. These images may have medical diagnostic value, e.g., in determining the state of the health of the tissue examined.

In a magnetic-resonance imaging process, a patient is typically aligned to place the portion of the patient's anatomy to be examined in the imaging volume of the magnetic-resonance imaging apparatus. Such an MRI device may comprise a primary magnet for supplying a constant magnetic field (Bo) which, by convention, may be along the z-axis and may be substantially homogeneous over the imaging volume, and secondary magnets that can provide linear magnetic field gradients along each of three principle Cartesian axes in space (e.g., x, y, and z, or x1, x2 and x3, respectively). A magnetic field gradient (ΔB₀/Δxi) refers to the variation of the field along the direction parallel to Bo with respect to each of the three principle Cartesian axes, xi. The MRI device may also comprise one or more radio-frequency (RF) coils, which provide excitation signals to the patient's body, placed in the imaging volume, in the form of a pulsed rotating magnetic field. This field may be referred to as the scanner's “B 1” field and as the scanner's “RF” or “radio-frequency” field. The frequency of the excitation signals may be the frequency at which this magnetic field rotates. These coils may also be used for detection of the excited patient's body material magnetic-resonance imaging response signals.

The presence of an IMD having one or more electrically conductive portions within a patient undergoing MRI may present one or more issues. For example, an IMD, such as, an implantable cardiac device (ICD) or implantable neurostimulator (INS), may have one or more electrically conductive leads that allow the IMD to conduct electrical signals to and/or from electrodes located on the lead(s). RF excitation during MRI may generate magnetic fields to tilt the spins from equilibrium to produce MRI signals. Electric fields produced during MRI RF excitation may couple to the conductive leads of the IMD, which act as an antenna, and may be deposited at a conductor/tissue interface (e.g., the electrode surface), causing local temperature elevations. In some examples, this phenomenon may be referred to as “RF lead heating.” Additionally or alternatively, the coupling of electric fields to the conductive leads may induce a current that may flow through the IMD. Such currents may cause heating and impact performance of the IMD.

In some examples, the power and/or duration of an MRI scan of a patient may be limited due to the presence of an IMD in a patient. In some examples, the power and/or duration of an MRI scan may be kept below a threshold to prevent an undesired amount of RF lead heating during the Mill scan and/or to prevent undesirable IMD performance. Such limitations on power and/or duration may reduce the quality of images generated by the Mill scan. In other examples, a clinician may elect to not perform an MRI scan on a patient with an implanted IMD to prevent the occurrence of such RF heating or IMD performance issues.

In accordance with one or more aspects of this disclosure, an IMD may include interconnect circuitry with an adjustable impedance such that the shunting impedance of the interconnect circuitry may be lowered during an Mill scan. The interconnect circuitry may interconnect an output of a stimulation generator with one or more electrodes. For instance, the interconnect circuitry may include one or more feedthrough capacitors; and one or more components and a switch that are collectively electrically connected in parallel with the feedthrough capacitor. Closing the switch places the one or more components in parallel with the feedthrough capacitor, reducing shunting impedance. When the switch is closed, the effective impedance of the parallel combination of the feedthrough capacitor and the one or more components may be lower than the effective impedance of the parallel combination of the feedthrough capacitor and the one or more components when the switch is open. The IMD may include processing circuitry configured to selectively close the switch based on a magnetic resonance imaging (MRI) status of the IMD. For instance, responsive to determining that the IMD is in an MRI mode or that MRI fields are detected, the processing circuitry may close the switch.

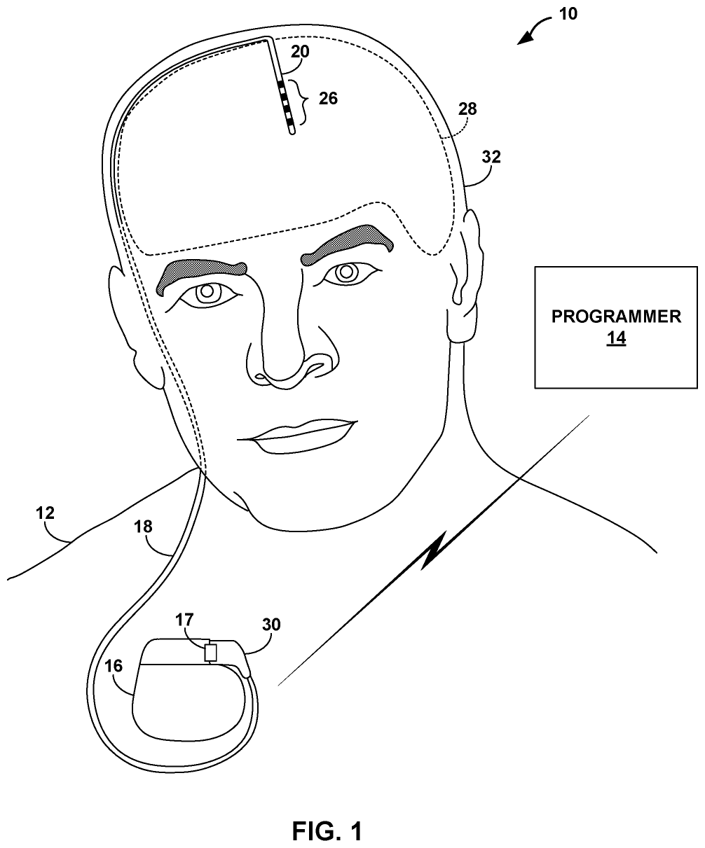

FIG. 1 is a conceptual diagram illustrating an example stimulation therapy system with a conductive lead implanted in the brain of a patient. It is noted that multiple conductive leads may be used in other examples. As shown in FIG. 1, system 10 includes IMD 16, lead extension 18, lead 20 and one or more electrodes 26 implanted within patient 12. Specifically, lead 20 enters through cranium 32 and is implanted within brain 28 to deliver DBS. One or more electrodes 26 of lead 20 provide electrical stimulation pulses to surrounding anatomical regions of brain 28 in a therapy that may treat or otherwise manage a condition of patient 12. In some examples, more than one lead 20 may be implanted within brain 28 of patient 12 to stimulate multiple anatomical regions of the brain. As shown in FIG. 1, system 10 may also include a programmer 14, which may be a handheld device, portable computer, or workstation that provides a user interface to a clinician. The clinician interacts with the user interface to program stimulation parameters.

For ease of illustration, examples of the disclosure will primarily be described with regard to implantable electrical stimulation leads and implantable medical devices that deliver neurostimulation therapy to a patient's brain in the form of DBS. However, the features and techniques described herein may be useful in other types of systems, including an IMD with one or more conductive leads or other conductive portions. For example, the features and techniques described herein may be used in systems with medical devices that deliver stimulation therapy to a patient's heart, e.g., implantable cardiac devices (ICDs) such as pacemakers, and pacemaker-cardioverter-defibrillators. As other examples, the features and techniques described herein may be embodied in systems that deliver other types of neurostimulation therapy (e.g., spinal cord stimulation, peripheral nerve stimulation, pelvic floor stimulation, such as sacral nerve stimulation), stimulation of at least one muscle or muscle groups, stimulation of at least one organ such as gastric system stimulation, stimulation concomitant to gene therapy, and, in general, stimulation of any tissue of a patient. As other examples, the features and techniques described herein may be embodied in systems that sense electrical signals to inform therapy or as diagnostic, such as, bioelectrical signals of the patient with or without stimulation capability.

Therapy system 10 includes medical device programmer 14, IMD 16, lead extension 18, and lead 20 with set of electrodes 26. IMD 16 includes a stimulation therapy module that includes an electrical stimulation generator that generates and delivers electrical stimulation therapy to one or more regions of brain 28 of patient 12 via one or more of electrode 26 of lead 20. In the example shown in FIG. 1, therapy system 10 may be referred to as a DBS system because IMD 16 provides electrical stimulation therapy directly to tissue within brain 28, e.g., a tissue site under the dura mater of brain 28. In other examples, leads 20 may be positioned to deliver therapy to a surface of brain 28 (e.g., the cortical surface of brain 28).

In the example shown in FIG. 1, IMD 16 may be implanted within a subcutaneous pocket above the clavicle of patient 12. In other examples, IMD 16 may be implanted within other regions of patient 12, such as a subcutaneous pocket in the abdomen or buttocks of patient 12 or proximate the cranium of patient 12. Implanted lead extension 18 is coupled to IMD 16 via connector block 30 (also referred to as a header), which may include, for example, electrical contacts that electrically couple to respective electrical contacts on lead extension 18. The electrical contacts electrically couple the electrodes 26 carried by lead 20 to IMD 16. Lead extension 18 traverses from the implant site of IMD 16 within a chest cavity of patient 12, along the neck of patient 12 and through the cranium of patient 12 to access brain 28. Generally, IMD 16 is constructed of a biocompatible material that resists corrosion and degradation from bodily fluids. IMD 16 may comprise a hermetic housing to substantially enclose components, such as a processor, therapy module, and memory. While shown in FIG. 1 as including leads, it is noted that the techniques of this disclosure are similarly applicable to lead-less devices.

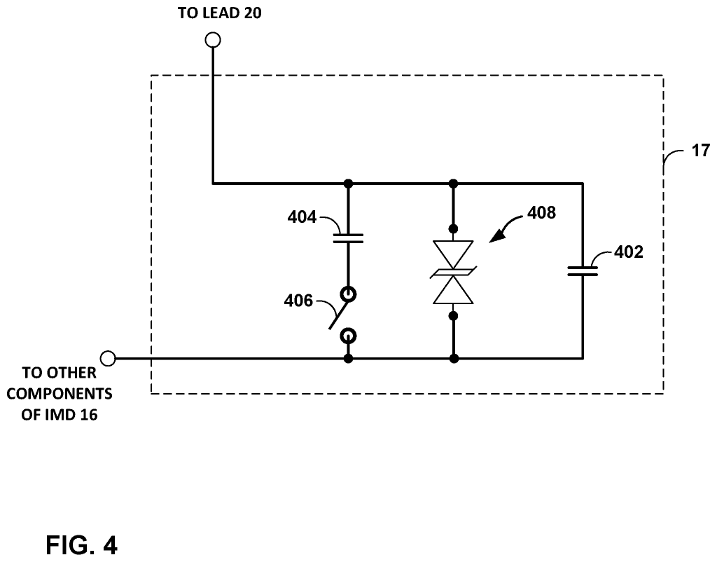

Signals traveling between IMD 16 and lead 20 may traverse interconnect circuitry 17. In some examples, interconnect circuitry 17 may include one or more feedthrough components, such as a feedthrough capacitor, that transport the electrical signals through the housing of IMD 16. In accordance with one or more techniques of this disclosure, an impedance of interconnect circuitry 17 may be adjustable between at least two levels. For instance, in a first operating mode (e.g., a normal operating mode) one or more components of interconnect circuitry 17 may be “switched out” such that the overall impedance of interconnect circuitry is a first level associated with a first impedance path. In a second operating mode (e.g., an MRI mode), the one or more components of interconnect circuitry 17 may be “switched in,” e.g., as a second impedance path in parallel with a first impedance path, such that the overall impedance of interconnect circuitry 17 is a second level that is less than the first level. Further details of one example of interconnect circuitry 17 are discussed below with reference to FIG. 4.

Lead 20 may be positioned to deliver electrical stimulation to one or more target tissue sites within brain 28 to manage patient symptoms associated with a disorder of patient 12. Lead 20 may be implanted to position electrodes 26 at desired locations of brain 28 through respective holes in cranium 32. Lead 20 may be placed at any location within brain 28 such that electrodes 26 are capable of providing electrical stimulation to target tissue sites within brain 28 during treatment. Although FIG. 1 illustrates system 10 as including one lead 20 coupled to IMD 16 via lead extension 18, in some examples, system 10 may include more than one lead.

Lead 20 may deliver electrical stimulation via electrodes 26 to treat any number of neurological disorders or diseases, such as movement disorders, seizure disorders or psychiatric disorders. Lead 20 may be implanted within a desired location of brain 28 via any suitable technique, such as through respective burr holes in a skull of patient 12 or through a common burr hole in the cranium 32. Lead 20 may be placed at any location within brain 28 such that electrodes 26 of lead 20 are capable of providing electrical stimulation to targeted tissue during treatment. In the examples shown in FIG. 1, electrodes 26 of lead 20 are shown as ring electrodes. In other examples, electrodes 26 of lead 20 may have different configurations including segmented electrodes or paddle electrodes. Electrodes 26 of lead 20 may have a complex electrode array geometry that is capable of producing shaped electrical fields. In this manner, e.g., with segmented electrodes or complex electrode geometries, electrical stimulation may be directed to a specific direction from lead 20 to enhance therapy efficacy and reduce possible adverse side effects from stimulating a large volume of tissue.

IMD 16 may deliver electrical stimulation therapy to brain 28 of patient 12 according to one or more stimulation therapy programs. A therapy program may define one or more electrical stimulation parameter values for therapy generated and delivered from IMD 16 to brain 28 of patient 12. Where IMD 16 delivers electrical stimulation in the form of electrical pulses, for example, the stimulation therapy may be characterized by selected pulse parameters, such as pulse amplitude, pulse rate, and pulse width. In addition, if different electrodes are available for delivery of stimulation, the therapy may be further characterized by different electrode combinations, which can include selected electrodes and their respective polarities. The exact therapy parameter values of the stimulation therapy that helps manage or treat a patient disorder may be specific for the particular target stimulation site (e.g., the region of the brain) involved as well as the particular patient and patient condition.

In addition to delivering therapy to manage a disorder of patient 12, therapy system 10 monitors one or more bioelectrical brain signals of patient 12. For example, IMD 16 may include a sensing module that senses bioelectrical brain signals within one or more regions of brain 28. In the example shown in FIG. 1, the signals generated by electrodes 26 are conducted to the sensing module within IMD 16 via conductors within lead 20 and lead extension 18. In some examples, a processor of IMD 16 may sense the bioelectrical signals within brain 28 of patient 12 and control delivery of electrical stimulation therapy to brain 28 via electrodes 26 in response to or based on the sensed bioelectrical signals. The bioelectrical brain signals monitored by IMD 16 may reflect changes in electrical current produced by the sum of electrical potential differences across brain tissue. Examples of the monitored bioelectrical brain signals include, but are not limited to, an electroencephalogram (EEG) signal, an electrocorticogram (ECoG) signal, a local field potential (LFP) sensed from within one or more regions of a patient's brain and/or action potentials from single cells within the patient's brain.

External programmer 14 wirelessly communicates with IMD 16 as needed to provide or retrieve therapy information. Programmer 14 is an external computing device that the user, e.g., the clinician and/or patient 12, may use to communicate with IMD 16. For example, programmer 14 may be a clinician programmer that the clinician uses to communicate with IMD 16 and program one or more therapy programs for IMD 16. Alternatively, programmer 14 may be a patient programmer that allows patient 12 to select programs and/or view and modify therapy parameters. The clinician programmer may include more programming features than the patient programmer. In other words, more complex or sensitive tasks may only be allowed by the clinician programmer to prevent an untrained patient from making undesired changes to IMD 16. In some examples, external programmer 14 may send a message, signal or command to IMD 16 that causes IMD 16 to operate in a different mode. For instance, an MRI technician may use external programmer 14 to send a message, signal or command to IMD 16 that causes IMD 16 to operate in the MRI mode (e.g., in which the impedance of interconnect circuitry 17 is adjusted).

Programmer 14 may be a hand-held computing device with a display viewable by the user and an interface for providing input to programmer 14 (i.e., a user input mechanism). In other examples, programmer 14 may be a larger workstation or a separate application within another multi-function device, rather than a dedicated computing device. For example, the multi-function device may be a notebook computer, tablet computer, workstation, cellular phone, personal digital assistant or another computing device that may run an application that enables the computing device to operate as a secure medical device programmer 14.

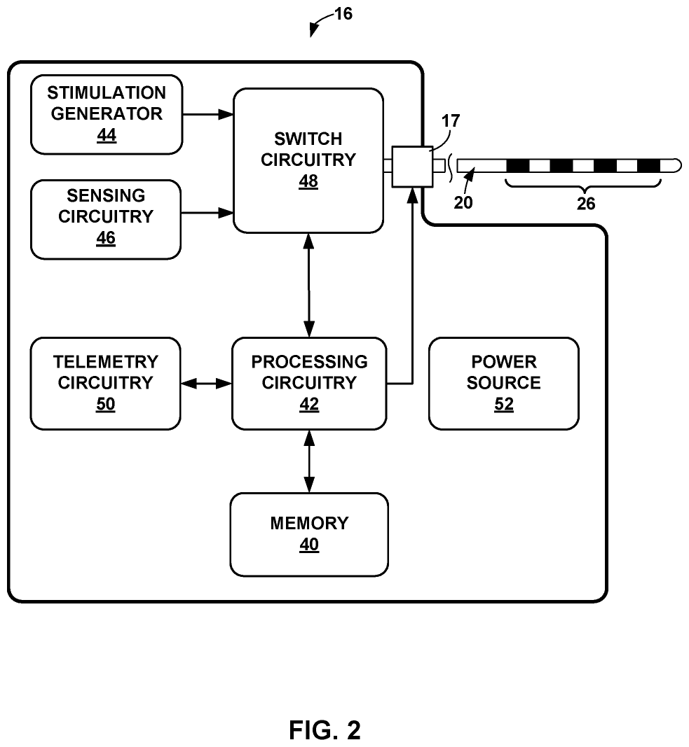

FIG. 2 is a functional block diagram illustrating components of IMD 16. In the example shown in FIG. 2, IMD 16 includes memory 40, processing circuitry 42, stimulation generator 44, sensing circuitry 46, switch circuitry 48, telemetry circuitry 50, and power source 52. Processing circuitry 42 may include any one or more microprocessors, controllers, digital signal processors (DSPs), application specific integrated circuits (ASICs), field-programmable gate arrays (FPGAs), or discrete logic circuitry. The functions attributed to processors described herein, including processing circuitry 42, may be provided by processing circuitry of a hardware device, e.g., as supported by software and/or firmware.

In the example shown in FIG. 2, sensing circuitry 46 may sense bioelectrical brain signals of patient 12 via select combinations of electrodes 26. The output of sensing circuitry 46 may be received by processing circuitry 42. In some cases, processing circuitry 42 may apply additional processing to the bioelectrical signals, e.g., convert the output to digital values for processing and/or amplify the bioelectrical brain signal. In addition, in some examples, sensing circuitry 46 or processing circuitry 42 may filter the signal from the selected electrodes 26 in order to remove undesirable artifacts from the signal, such as noise from cardiac signals generated within the body of patient 12. Although sensing circuitry 46 is incorporated into a common outer housing with stimulation generator 44 and processing circuitry 42 in FIG. 2, in other examples, sensing circuitry 46 is in a separate outer housing from the outer housing of IMD 16 and communicates with processing circuitry 42 via wired or wireless communication techniques. In some examples, sensing circuitry 46 may sense brain signals substantially at the same time that IMD 16 delivers therapy to patient 12. In other examples, sensing circuitry 46 may sense brain signals and IMD 16 may deliver therapy at different times.

Memory 40 may include any volatile or non-volatile media, such as a random access memory (RAM), read only memory (ROM), non-volatile RAM (NVRAM), electrically erasable programmable ROM (EEPROM), flash memory, and the like. Memory 40 may store computer-readable instructions that, when executed by processing circuitry 42, cause IMD 16 to perform various functions described herein. Memory 40 may be considered, in some examples, a non-transitory computer-readable data storage medium comprising instructions that cause one or more processors, such as, e.g., processing circuitry 42, to implement one or more of the example techniques described in this disclosure. The term “non-transitory” may indicate that the data storage medium is not embodied in a carrier wave or a propagated signal. However, the term “non-transitory” should not be interpreted to mean that memory 40 is non-movable. As one example, memory 40 may be removed from IMD 16, and moved to another device. In certain examples, a non-transitory storage medium may store data that can, over time, change (e.g., in RAM).

In the example shown in FIG. 2, processing circuitry 42 controls switch circuitry 48 to sense bioelectrical brain signals with selected combinations of electrodes 26. For example, switch circuitry 48 may create or cut off electrical connections between sensing circuitry 46 and selected electrodes 26 in order to selectively sense bioelectrical brain signals, e.g., in particular portions of brain 28 of patient 12. Processing circuitry 42 may also control switch circuitry 48 to apply stimulation signals generated by stimulation generator 44 to selected combinations of electrodes 26. In particular, switch circuitry 48 may couple stimulation signals to selected conductors within leads 20, which, in turn, deliver the stimulation signals across selected electrodes 26. Switch circuitry 48 may be a switch array, switch matrix, multiplexer, or any other type of switching module configured to selectively couple stimulation energy to selected electrodes 26 and to selectively sense bioelectrical brain signals with selected electrodes 26. Hence, stimulation generator 44 is coupled to electrodes 26 via switch circuitry 48 and conductors within leads 20. In some examples, however, IMD 16 does not include switch circuitry 48. In some examples, IMD 16 may include separate current sources and sinks for each individual electrode (e.g., instead of a single stimulation generator) such that switch circuitry 48 may not be necessary.

In some examples, IMD 16 may be considered to be a stimulate-only device. For instance, IMD 16 may omit sensing circuitry 46. In some examples, IMD 16 may be considered to be a sense-only device. For instance, IMD 16 may omit stimulation generator 44.

As discussed above, interconnect circuitry 17 of IMD 16 may include components through which signals traveling between IMD 16 and lead 20 may traverse. For instance, as shown in FIG. 2, signals between electrodes 26 of lead 20 and stimulation generator 44/sensing circuitry 46 may traverse interconnect circuitry 17. In some examples, interconnect circuitry 17 may include one or more feedthrough components, such as a feedthrough capacitor, that transport the electrical signals through the housing of IMD 16 (e.g., as shown in FIG. 2 where interconnect circuitry is shown as crossing a boundary of IMD 16). In accordance with one or more techniques of this disclosure, an impedance of interconnect circuitry 17 may be adjustable between at least two levels. For instance, in a first operating mode (e.g., a normal operating mode) one or more components of interconnect circuitry 17 may be “switched out” such that the overall impedance of interconnect circuitry is a first level. In a second operating mode (e.g., an MRI mode), the one or more components of interconnect circuitry 17 may be “switched in” such that the overall impedance of interconnect circuitry is a second level that is lower than the first level. As shown in FIG. 2, operation of interconnect circuitry 17 (e.g., of a switch that connects the one or more components) may be controlled by processing circuitry 42. Further details of one example of interconnect circuitry 17 are discussed below with reference to FIG. 4.

Stimulation generator 44 may be a single channel or multi-channel stimulation generator. For example, stimulation generator 44 may be capable of delivering, a single stimulation pulse, multiple stimulation pulses or a continuous signal at a given time via a single electrode combination or multiple stimulation pulses at a given time via multiple electrode combinations, where each electrode combination includes two or more electrodes arranged, e.g., in a unipolar, bipolar or multipolar configuration. In some examples, however, stimulation generator 44 and switch circuitry 48 may be configured to deliver multiple channels of stimulation pulses on a time-interleaved basis. For example, switch circuitry 48 may serve to time divide the output of stimulation generator 44 across different electrode combinations at different times to deliver multiple programs or channels of stimulation energy to patient 12.

Telemetry circuitry 50 may support wireless communication between IMD 16 and an external programmer 14 or another computing device under the control of processing circuitry 42. Telemetry circuitry 50 in IMD 16, as well as telemetry modules in other devices and systems described herein, such as programmer 14, may accomplish communication by radiofrequency (RF) communication techniques. In addition, telemetry circuitry 50 may communicate with external programmer 14 via proximal inductive interaction of IMD 16 with programmer 14. Accordingly, telemetry circuitry 50 may send information to external programmer 14 on a continuous basis, at periodic intervals, or upon request from IMD 16 or programmer 14.

In some examples, telemetry circuitry 50 may support wireless communication between IMD 16 and an MRI system under the control of processing circuitry 42, either directly or indirectly (e.g., via an intermediate device such as programmer 14). For example, using telemetry circuitry 50, processing circuitry 42 may transmit information regarding the voltage induced by an RF field of an MRI scanner, e.g., during an MRI scan, and sensed via sensing circuitry 46. In some examples, IMD 16 may transmit raw or processed signal information (e.g., the determined Vrms of the sensed induced voltage) to the MRI system directly or indirectly. Based on the received information from IMD 16, the MRI system may make one or more adjustments to an MRI scan performed on patient 12. Additionally, or alternatively, IMD 16 may sense the induced voltage and determine one or more adjustments to be made to an MRI scan based on the sensed induced voltage, and then transmit instructions to the MRI system with the one or more adjustments to the MRI scan for implementation by the MRI system.

Power source 52 delivers operating power to various components of IMD 16. Power source 52 may include a small rechargeable or non-rechargeable battery and a power generation circuit to produce the operating power. Recharging may be accomplished through proximal inductive interaction between an external charger and an inductive charging coil within IMD 16. In some examples, power requirements may be small enough to allow IMD 16 to utilize patient motion and implement a kinetic energy-scavenging device to trickle charge a rechargeable battery. In other examples, traditional batteries may be used for a limited period of time.

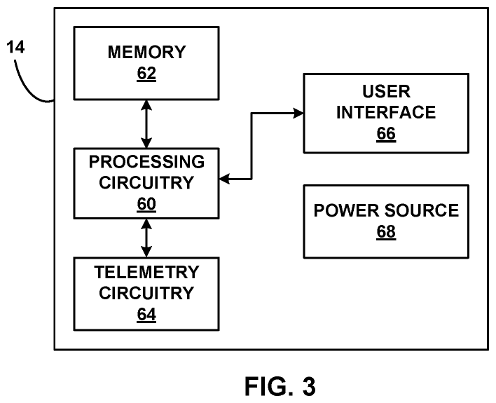

FIG. 3 is a conceptual block diagram of an example external medical device programmer 14, which includes processing circuitry 60, memory 62, telemetry circuitry 64, user interface 66, and power source 68. Processing circuitry 60 controls user interface 66 and telemetry circuitry 64, and stores and retrieves information and instructions to and from memory 62. Programmer 14 may be configured for use as a clinician programmer or a patient programmer. Processing circuitry 60 may comprise any combination of one or more processors including one or more microprocessors, DSPs, ASICs, FPGAs, or other equivalent integrated or discrete logic circuitry. Accordingly, processing circuitry 60 may include any suitable structure, whether in hardware, software, firmware, or any combination thereof, to perform the functions ascribed herein to processing circuitry 60.

Memory 62 may include instructions for operating user interface 66 and telemetry circuitry 64, and for managing power source 68. Memory 62 may also store any therapy data retrieved from IMD 16 during the course of therapy. Memory 62 may include any volatile or nonvolatile memory, such as RAM, ROM, EEPROM or flash memory. Memory 62 may also include a removable memory portion that may be used to provide memory updates or increases in memory capacities. A removable memory may also allow sensitive patient data to be removed before programmer 14 is used by a different patient. Memory 62 may be considered, in some examples, a non-transitory computer-readable data storage medium comprising instructions that cause one or more processors, such as, e.g., processing circuitry 60, to implement one or more of the example techniques described in this disclosure. The term “non-transitory” may indicate that the data storage medium is not embodied in a carrier wave or a propagated signal. However, the term “non-transitory” should not be interpreted to mean that memory 62 is non-movable. As one example, memory 62 may be removed from programmer 14, and moved to another device. In certain examples, a non-transitory storage medium may store data that can, over time, change (e.g., in RAM).

Wireless telemetry in programmer 14 may be accomplished by RF communication or proximal inductive interaction of external programmer 14 with IMD 16. This wireless communication is possible through the use of telemetry circuitry 64. Accordingly, telemetry circuitry 64 may be similar to the telemetry circuitry contained within IMD 16. In alternative examples, programmer 14 may be capable of infrared communication or direct communication through a wired connection. In this manner, other external devices may be capable of communicating with programmer 14 without needing to establish a secure wireless connection.

Power source 68 may deliver operating power to the components of programmer 14. Power source 68 may include a battery and a power generation circuit to produce the operating power. In some examples, the battery may be rechargeable to allow extended operation.

In some examples, programmer 14 may be configured to communicate with IMD 16 to change or adjust an operating mode of IMD 16. For instance, user interface 66 may receive user input requesting that IMD 16 operate in an MRI mode (e.g., an MRI safe mode). Responsive to receiving such user input via user interface 66, processing circuitry 60 may output, via telemetry circuitry 64, a message to IMD 16 that causes IMD 16 to transition to operating in the MRI mode. As discussed herein, when operating in the MRI mode, IMD 16 may adjust an impedance between lead 20 and one or more components of IMD 16 (e.g., stimulation generator 44 and/or sensing circuitry 46).

FIG. 4 is a schematic diagram illustrating one example of interconnect circuitry with adjustable impedance, in accordance with one or more aspects of this disclosure. As shown in FIG. 4, interconnect circuitry 17 may include, feedthrough capacitor 402, supplemental capacitor 404, switch 406, and diodes 408. As also shown in FIG. 4, a first terminal of interconnect circuitry 17 may be electrically connected to other components of IMD 16, such as switch circuitry 48, and a second terminal of interconnect circuitry 17 may be electrically connected to lead 20.

Feedthrough capacitor 402 may form a portion of an electrical feedthrough that carries electrical signals between components inside a housing of IMD 16 and components outside the housing. By including feedthrough capacitor 402 as opposed to a direct electrical connection, electromagnetic interference (EMI) entering the housing of IMD 16 may be reduced.

Diodes 408 may provide for additional transient voltage suppression through interconnect circuitry 17. For instance, large voltage spikes picked up on electrodes are clamped to lower levels to protect internal components of IMD 16. In some examples, diodes 408 may be referred to as defib (defibrillation) diodes.

As discussed above, there are competing reasons for having a low shunting impedance of interconnect circuitry 17 and having a large shunting impedance of interconnect circuitry 17. For instance, low shunt impedances of interconnect circuitry 17 may result in relatively low heating of components of IMD 16 while IMD 16 is subjected to an MRI field while higher shunt impedances of interconnect circuitry 17 may result in relatively high heating of IMD 16 while IMD 16 is subjected to an MRI field. Therefore, it is desirable for interconnect circuitry 17 to have a relatively lower shunt impedance. However, lower shunt impedances of interconnect circuitry 17 may present one or more disadvantages. As one example, the impedance of interconnect circuitry 17 may have a negative relationship with an amount of energy used to deliver a particular level of stimulation. For instance, lower shunt impedances may result in an increased amount of energy (e.g., battery power) being expended by stimulation generator 44 to deliver a particular level of stimulation as compared with higher shunt impedances. Therefore, it is desirable for interconnect circuitry 17 to have a relatively higher shunt impedance. As another example, the impedance of interconnect circuitry 17 may have a positive relationship with a quality of sensing by sensing circuitry 46. For instance, lower shunt impedances may result in a decrease in common-mode signal rejection as compared with higher shunt impedances due to component mismatch tolerances. Therefore, it is desirable for interconnect circuitry 17 to have a relatively higher shunt impedance.

In accordance with one or more aspects of this disclosure, interconnect circuitry 17 may include a dynamically adjustable impedance. For instance, interconnect circuitry 17 may include one or more components and a switch that are collectively electrically connected in parallel with the feedthrough capacitor. As shown in the example of FIG. 4, the one or more components may include a capacitor such as capacitor 404 and the switch may be switch 406. While illustrated and described as a capacitor, the one or more components may be any type of circuit element. For instance, capacitor 404 may be replaced by a resistor or an inductor. Additionally, while illustrated as a single capacitor, capacitor 404 may be a plurality of capacitors in series or parallel (e.g., with an effective capacitance that can be switched in parallel with the feedthrough capacitor).

An impedance of the one or more components (e.g., capacitor 404) may be lower than an impedance of feedthrough capacitor 402. In some examples, the capacitance of the one or more components may be at least one order of magnitude larger than the capacitance of capacitor 402. For instance, in one example, capacitor 402 may have a capacitance of approximately 65 pico farads (pF), presenting a relatively low capacitance in a first mode when switch 406 is open, and capacitor 404 may have an capacitance of at least approximately 650 pF, such that the combined capacitance of capacitors 402 and 404 present a relatively higher capacitance (e.g., 705 pF) in a second mode when switch 406 is closed. In some examples, the capacitance of the one or more components may be at least N (e.g., 5, 10, 15, 20, 25, etc.) times the capacitance of capacitor 404. For instance, where N is 20, capacitor 404 may have a capacitance of 65 pico farads (pF) and capacitor 404 may have an impedance of at least 1300 pF. In some examples, the capacitance of the feedthrough 402 may effectively zero or just be the parasitic capacitance of the circuit, and the switched in capacitance 404 may be the totality of feedthrough capacitance of 1000 pF to 2000 pF.

As the effective capacitance of two capacitors in parallel is the sum of the capacitors, the effective capacitance of capacitors 402 and 404 may be toggled between a first mode in which the capacitance of interconnect circuitry 17 is provided by capacitor 402 (and not capacitor 404) when switch 406 is open and a second mode in which the capacitance of interconnect circuitry 17 is provided by the combined capacitance of capacitors 402 and 404 when switch 406 is closed. In this way, the impedance of interconnect circuitry 17 may be considered to be dynamic.

As discussed above, operation of interconnect circuitry 17 may be controlled by one or more other components of IMD 16, such as processing circuitry 42. For instance, processing circuitry 42 may be configured to selectively operate switch 406 based on a magnetic resonance imaging (MRI) status of the IMD. As one example, processing circuitry 42 may be configured to close switch 406 responsive to determining that IMD 16 is operating in an MRI mode. As another example, processing circuitry 42 may be configured to open switch 406 responsive to determining that IMB 16 is operating in a normal mode. In this way, interconnect circuitry 17 may present a smaller shunt impedance when IMD 16 is operating in the MRI mode than when IMB 16 is operating in the normal mode.

As shown in FIG. 4, switch 406 may be a low-side switch relative to the one or more components. For instance, switch 406 may be positioned between capacitor 404 and the connection to lead 20. In other examples, switch 406 may be a high-side switch relative to the one or more components. For instance, switch 406 may be positioned between capacitor 404 and the connection to lead 20.

In some examples, additional components may be located between interconnect circuitry 17 and the other components of IMD 16. For instance, a capacitor may be located at the “other components” terminal of interconnect circuitry 17.

In some examples, IMD 16 may include multiple “copies” of interconnect circuitry 17, which may be referred to as channels. For instance, IMD 16 may include a separate channel for each lead included on a lead connected to IMD 16 (e.g., a separate copy of interconnect circuitry 17 for each of electrodes 26 of lead 20).

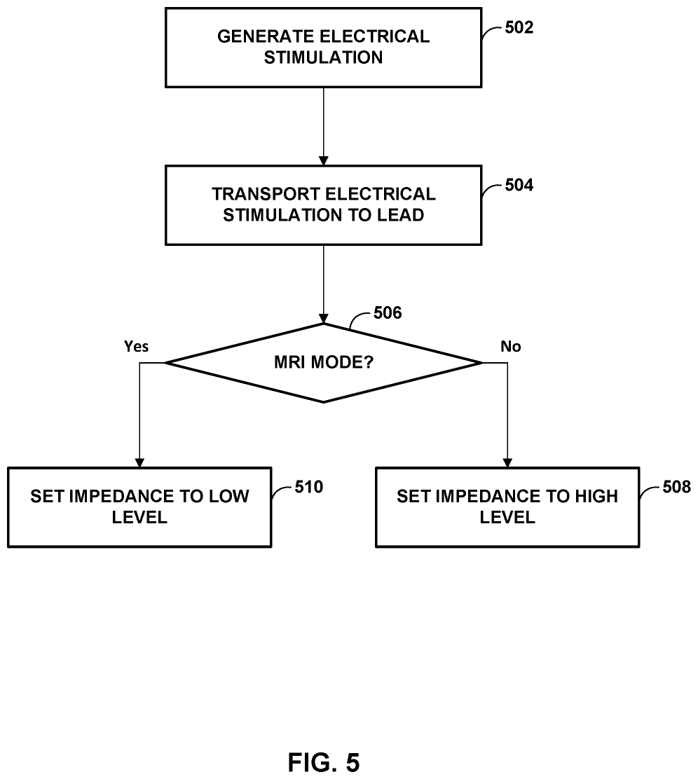

FIG. 5 is a flow diagram illustrating an example technique for dynamically adjusting an impedance of an implantable medical device, in accordance with one or more aspects of this disclosure. For ease of description, the example technique of FIG. 5 is described with regard to IMD 16 of FIG. 2. However, any suitable system including an IMD may be employed to perform the example technique of FIG.5.

As shown in FIG. 5, IMD 16 may generate electrical stimulation (502). For instance, simulation generator 44 may generate electrical stimulation for delivery to a patient via lead 20. The electrical stimulation may be in the form of electrical pulses.

IMD 16 may transport the electrical stimulation to a lead (504). For instance, switch circuitry 48 and interconnect circuitry 17 may transport the electrical stimulation generated by stimulation generator 44 to lead 20. As discussed above, interconnect circuitry 17 may include a feedthrough capacitor (e.g., feedthrough capacitor 402); and one or more components and a switch that are collectively electrically connected in parallel with the feedthrough capacitor (e.g., capacitor 404 and switch 406).

IMD 16 may dynamically adjust an impedance based on an MRI status of IMD 16. As one example, responsive to determining that IMD 16 is in an MRI mode (“Yes” branch of 506), IMD 16 may set the impedance to a low level (510). For instance, processing circuitry 42 may output a signal that causes switch 406 to close. As another example, responsive to determining that IMD 16 is in a normal mode (“No” branch of 506), IMD 16 may set the impedance to a high level (508). For instance, processing circuitry 42 may output a signal that causes switch 406 to open. As discussed above, when switch 406 is closed, the impedance of interconnect circuitry 17 may be higher than when switch 406 is open.

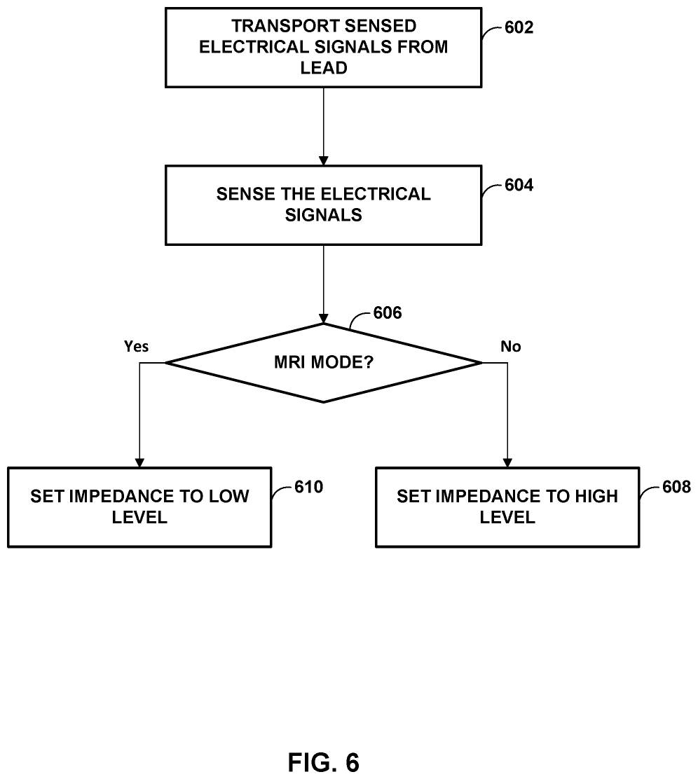

FIG. 6 is a flow diagram illustrating an example technique for dynamically adjusting an impedance of an implantable medical device, in accordance with one or more aspects of this disclosure. For ease of description, the example technique of FIG. 6 is described with regard to IMD 16 of FIG. 2. However, any suitable system including an IMD may be employed to perform the example technique of FIG.5.

As shown in FIG. 6, IMD 16 may transport electrical signals from a lead (602). For instance, switch circuitry 48 and interconnect circuitry 17 may transport the electrical signals received via lead to sensing circuitry 46. As discussed above, interconnect circuitry 17 may include a feedthrough capacitor (e.g., feedthrough capacitor 402); and one or more components and a switch that are collectively electrically connected in parallel with the feedthrough capacitor (e.g., capacitor 404 and switch 406).

IMD 16 may sense the electrical signals (604). For instance, sensing circuitry 46 may sense one or more signals, such as local field potentials (LFP) and/or evoked compound action potentials (ECAP) in the electrical signals.

IMD 16 may dynamically adjust an impedance based on an MRI status of IMD 16. As one example, responsive to determining that IMD 16 is in an MRI mode (“Yes” branch of 606), IMD 16 may set the impedance to a low level (610). For instance, processing circuitry 42 may output a signal that causes switch 406 to close. As another example, responsive to determining that IMD 16 is in a normal mode (“No” branch of 606), IMD 16 may set the impedance to a high level (608). For instance, processing circuitry 42 may output a signal that causes switch 406 to open. As discussed above, when switch 406 is closed, the impedance of interconnect circuitry 17 may be higher than when switch 406 is open.

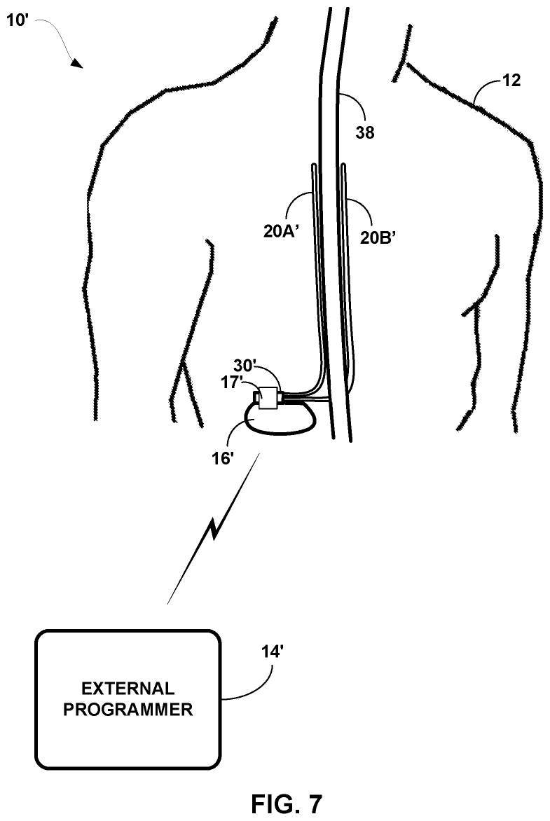

FIG. 7 is a conceptual diagram illustrating an example medical device system. As shown in FIG. 1, the techniques of disclosure are applicable to implantable medical devices used for brain stimulation. As shown in FIG. 7, the techniques of this disclosure are similarly applicable to implantable medical devices used for spinal cord stimulation. For instance, as shown in FIG. 7, leads 20A′ and 20B′ may be implanted proximal to spinal cord 38 of patient 12. IMD 16′, interconnect circuitry 17′, leads 20A′ and 20B′, and connector block 30′ of system 10′ of FIG. 7 may be considered to perform operations similar to IMD 16, interconnect circuitry 17, leads 20A and 20B, and connector block 30 of system 10 of FIG. 1.

In operation, IMD 16′ may deliver stimulation to and/or perform sensing on signals received from spine 38 via leads 20A′ and 20B′. Some signals that may sensed include, but are not limited to, one of an electroencephalogram (EEG) signal, an electrocorticogram (ECoG) signal, a local field potential (LFP), or an evoked compound action potential (ECAP) signal. As discussed above, it may be desirable for interconnect circuitry 17′ to have a high shunt impedance when IMD 16′ is delivering stimulation and/or sensing but have a low shunt impedance when IMD 16′ is or may be subject to MRI fields. In accordance with one or more aspects of this disclosure, interconnect circuitry 17′ may have an adjustable impedance. For instance, interconnect circuitry 17′ may include one or more feedthrough capacitors; and one or more components and a switch that are collectively electrically connected in parallel with the one of more feedthrough capacitors. Processing circuitry of IMD 16′ may selectively close the switch based on the MRI status of the IMD to place the one or more components in parallel with the one or more feedthrough capacitor.

The following numbered examples may illustrate one or more aspects of this disclosure:

Clause 1. A system comprising an implantable medical device (IMD), the IMD comprising: a stimulation generator configured to generate electrical stimulation for delivery to a patient via a lead coupled to the IMD; interconnect circuitry configured to deliver the electrical stimulation from the stimulation generator to the lead; and processing circuitry configured to adjust an impedance of the interconnect circuitry based on a magnetic resonance imaging (MRI) status of the IMD.

Clause 2. The system of clause 1, wherein the interconnect circuitry comprises: one or more feedthrough capacitors; and one or more components and a switch that are collectively electrically connected in parallel with the one of more feedthrough capacitors, wherein the processing circuitry is configured to selectively close the switch based on the MRI status of the IMD to place the one or more components in parallel with the one or more feedthrough capacitors.

Clause 3. The system of clause 2, wherein the one or more components comprise a second capacitor, and wherein a capacitance of the second capacitor is at least one order of magnitude larger than a capacitance of the feedthrough capacitor.

Clause 4. The system of clause 2 or 3, wherein the switch comprises a low-side switch relative to the one or more components.

Clause 5. The system of any of clauses 1-4, wherein the IMD further comprises: sensing circuitry configured to sense electrical signals at one or more electrodes of the lead via the interconnect circuitry.

Clause 6. The system of clause 5, wherein the electrical signals include one of an electroencephalogram (EEG) signal, an electrocorticogram (ECoG) signal, a local field potential (LFP), electromyogram (EMG), or an evoked compound action potential (ECAP) signal.

Clause 7. The system of any of clauses 1-6, wherein, to adjust the impedance based on the MRI status, the processing circuitry is configured to: adjust the impedance to a first impedance level responsive to determining that the IMD is operating in an MRI mode; and adjust the impedance to a second impedance level responsive to determining that the IMD is operating in a normal mode, the first impedance level being lower than the second impedance level.

Clause 8. The system of any of clauses 1-7, wherein, to selectively close the switch based on the MRI status, the processing circuitry is configured to: close the switch responsive to detecting an MRI field.

Clause 9. The system of any of clauses 1-8, further comprising the lead, wherein the lead comprises one or more electrodes via which the electrical stimulation is delivered.

Clause 10. A method comprising: generating, by a stimulation generator of an implantable medical device (IMD), electrical stimulation for delivery to a patient via a lead coupled to the IMD; transporting, by interconnect circuitry, the electrical stimulation from the stimulation generator to the lead; and adjusting, by processing circuitry, an impedance of the interconnect circuitry based on a magnetic resonance imaging (MRI) status of the IMD.

Clause 11. The method of clause 10, further comprising: receiving, by sensing circuitry of the IMD and via the interconnect circuitry, electrical levels sensed at one or more electrodes of the lead.

Clause 12. The method of cause 10 or 11, wherein the interconnect circuitry comprises: a feedthrough capacitor; and one or more components and a switch that are collectively electrically connected in parallel with the feedthrough capacitor; and wherein adjusting the impedance comprises operating the switch based on the MRI status of the IMD.

Clause 13. The method of clause 12, wherein selectively operating the switch comprises: closing the switch responsive to determining that the IMD is operating in an Mill mode; and opening the switch responsive to determining that the IMD is operating in a normal mode.

Clause 14. The method of any of clauses 10-13, further comprising: operating the IMD in the MRI mode responsive to detecting an Mill field.

Clause 15. A computer-readable storage medium storing instructions that, when executed, cause processing circuitry of an implantable medical device (IMD) to: cause a stimulation generator to generate electrical stimulation for delivery to a patient via a lead coupled to the IMD, the electrical stimulation being transported from the stimulation generator to the lead via interconnect circuitry; and adjust an impedance of the interconnect circuitry based on a magnetic resonance imaging (Mill) status of the IMD.

Clause 16. The computer-readable storage medium of clause 15, further comprising instructions that cause the processing circuitry to: cause sensing circuitry of the IMD to receive, via the interconnect circuitry, electrical levels sensed at one or more electrodes of the lead.

Clause 17. The computer-readable storage medium of clause 15 or 16, wherein the interconnect circuitry comprises: a feedthrough capacitor; and one or more components and a switch that are collectively electrically connected in parallel with the feedthrough capacitor; and wherein the instructions that cause the processing circuitry to adjust the impedance comprise instructions that cause the processing circuitry to selectively operate the switch based on the Mill status of the IMD.

Clause 18. The computer-readable storage medium of claim 17, wherein the instructions that cause the processing circuity to selectively operate the switch comprise instructions that cause the processing circuitry to: close the switch responsive to determining that the IMD is operating in an MRI mode; and open the switch responsive to determining that the IMD is operating in a normal mode.

Clause 19. The computer-readable storage medium of any of clauses 15-18, further comprising instructions that cause the processing circuitry to: operate the IMD in the MRI mode responsive to detecting an MRI field.

Clause 20. A system comprising an implantable medical device (IMD), the IMD comprising: sensing circuitry configured to sense electrical signals at one or more electrodes of a lead coupled to the IMD; interconnect circuitry configured to transport the electrical signals from the one or more electrodes to the sensing circuitry; and processing circuitry configured to adjust an impedance of the interconnect circuitry based on a magnetic resonance imaging (MRI) status of the IMD.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory. The computer-readable storage media may be referred to as non-transitory. A programmer, such as patient programmer or clinician programmer, or other computing device may also contain a more portable removable memory type to enable easy data transfer or offline data analysis.

As used herein, the term “circuitry” may refer to an ASIC, an electronic circuit, a processor (shared, dedicated, or group) and memory that execute one or more software or firmware programs, a combinational logic circuit, or other suitable components that provide the described functionality. The term “processing circuitry” refers one or more processors distributed across one or more devices. For example, “processing circuitry” can include a single processor or multiple processors on a device. “Processing circuitry” can also include processors on multiple devices, wherein the operations described herein may be distributed across the processors and devices.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

1. A system comprising an implantable medical device (IMD), the IMD comprising:
a stimulation generator configured to generate electrical stimulation for delivery to a patient via a lead coupled to the IMD;
interconnect circuitry configured to deliver the electrical stimulation from the stimulation generator to the lead; and
processing circuitry configured to adjust an impedance of the interconnect circuitry based on a magnetic resonance imaging (MRI) status of the IMD.

2. The system of claim 1, wherein the interconnect circuitry comprises:
one or more feedthrough capacitors; and
one or more components and a switch that are collectively electrically connected in parallel with the one of more feedthrough capacitors,
wherein the processing circuitry is configured to selectively close the switch based on the MRI status of the IMD to place the one or more components in parallel with the one or more feedthrough capacitors.

3. The system of claim 2, wherein the one or more components comprise a second capacitor, and wherein a capacitance of the second capacitor is at least one order of magnitude larger than a capacitance of the feedthrough capacitor.

4. The system of claim 2, wherein the switch comprises a low-side switch relative to the one or more components.

5. The system of claim 1, wherein the IMD further comprises:
sensing circuitry configured to sense electrical signals at one or more electrodes of the lead via the interconnect circuitry.

6. The system of claim 5, wherein the electrical signals include one of an electroencephalogram (EEG) signal, an electrocorticogram (ECoG) signal, a local field potential (LFP), electromyogram (EMG), or an evoked compound action potential (ECAP) signal.

7. The system of claim 1, wherein, to adjust the impedance based on the MRI status, the processing circuitry is configured to:
adjust the impedance to a first impedance level responsive to determining that the IMD is operating in an MRI mode; and
adjust the impedance to a second impedance level responsive to determining that the IMD is operating in a normal mode, the first impedance level being lower than the second impedance level.

8. The system of claim 1, wherein, to selectively close the switch based on the MRI status, the processing circuitry is configured to:
close the switch responsive to detecting an MRI field.

9. The system of claim 1, further comprising the lead, wherein the lead comprises one or more electrodes via which the electrical stimulation is delivered.

10. A method comprising:
generating, by a stimulation generator of an implantable medical device (IMD), electrical stimulation for delivery to a patient via a lead coupled to the IMD;
transporting, by interconnect circuitry, the electrical stimulation from the stimulation generator to the lead; and
adjusting, by processing circuitry, an impedance of the interconnect circuitry based on a magnetic resonance imaging (MRI) status of the IMD.

11. The method of claim 10, further comprising:
receiving, by sensing circuitry of the IMD and via the interconnect circuitry, electrical levels sensed at one or more electrodes of the lead.

12. The method of claim 10, wherein the interconnect circuitry comprises:
a feedthrough capacitor; and
one or more components and a switch that are collectively electrically connected in parallel with the feedthrough capacitor; and
wherein adjusting the impedance comprises operating the switch based on the MRI status of the 1MB.

13. The method of claim 12, wherein selectively operating the switch comprises:
closing the switch responsive to determining that the 1MB is operating in an MRI mode; and
opening the switch responsive to determining that the 1MB is operating in a normal mode.

14. The method of claim 10, further comprising:
operating the 1MB in the MRI mode responsive to detecting an MRI field.

15. A computer-readable storage medium storing instructions that, when executed, cause processing circuitry of an implantable medical device (IMD) to:
cause a stimulation generator to generate electrical stimulation for delivery to a patient via a lead coupled to the IMD, the electrical stimulation being transported from the stimulation generator to the lead via interconnect circuitry; and
adjust an impedance of the interconnect circuitry based on a magnetic resonance imaging (MRI) status of the IMD.

16. The computer-readable storage medium of claim 15, further comprising instructions that cause the processing circuitry to:
cause sensing circuitry of the IMD to receive, via the interconnect circuitry, electrical levels sensed at one or more electrodes of the lead.

17. The computer-readable storage medium of claim 15, wherein the interconnect circuitry comprises:
a feedthrough capacitor; and
one or more components and a switch that are collectively electrically connected in parallel with the feedthrough capacitor; and
wherein the instructions that cause the processing circuitry to adjust the impedance comprise instructions that cause the processing circuitry to selectively operate the switch based on the MRI status of the IMD.

18. The computer-readable storage medium of claim 17, wherein the instructions that cause the processing circuity to selectively operate the switch comprise instructions that cause the processing circuitry to:
close the switch responsive to determining that the IMD is operating in an MRI mode; and
open the switch responsive to determining that the IMD is operating in a normal mode.

19. The computer-readable storage medium of claim 15, further comprising instructions that cause the processing circuitry to:
operate the IMD in the Mill mode responsive to detecting an Mill field.

20. A system comprising an implantable medical device (IMD), the IMD comprising:
sensing circuitry configured to sense electrical signals at one or more electrodes of a lead coupled to the IMD;
interconnect circuitry configured to transport the electrical signals from the one or more electrodes to the sensing circuitry; and
processing circuitry configured to adjust an impedance of the interconnect circuitry based on a magnetic resonance imaging (Mill) status of the IMD.
